(19) United States
(12) Patent Application Publication
(10) Pub. No.: US 2025/0314566 A1
(43) Pub. Date: Oct. 9, 2025

(54) CALIBRATION CURVE SOLUTION PRODUCTION SYSTEM, MEASUREMENT SYSTEM, AND CALIBRATION CURVE SOLUTION PRODUCTION METHOD

(71) Applicant: ORGANO CORPORATION, Tokyo (JP)

(72) Inventors: Chiharu UMEZAWA, Tokyo (JP); Akihiko TSUDA, Tokyo (JP); Fumitaka ICHIHARA, Tokyo (JP)

(73) Assignee: ORGANO CORPORATION, Tokyo (JP)

(21) Appl. No.: 18/867,074

(22) PCT Filed: Mar. 15, 2023
(86) PCT No.: PCT/JP2023/010083

§ 371 Date: Nov. 19, 2024

(30) Foreign Application Priority Data
May 20, 2022 (JP) 2022-083008

(51) Int. Cl. G01N 1/38; G01N 1/14; G01N 33/18

(52) U.S. Cl. CPC G01N 1/38; G01N 1/14; G01N 33/18; G01N 2001/386

(57) ABSTRACT

A calibration curve solution production system that produces and supplies a calibration curve solution for creating a calibration curve to a measurement device that uses the calibration curve to measure the total organic carbon concentration of a liquid to be analyzed. The system includes an analyte line that conducts the liquid to be analyzed to a TOC analyzer, a standard solution line that conducts a standard solution to the analyte line, a mixing unit installed on the analyte line that mixes the diluent and the standard solution to dilute the standard solution and supplies to the TOC analyzer the mixture of the diluent and the standard solution mixed in mixing unit as the calibration curve solution.

### TECHNICAL FIELD

This invention relates to a calibration curve solution production system, a measurement system, and a calibration curve solution production method.

### BACKGROUND OF ART

In general, the concentration of total organic carbon (TOC) in liquids is controlled using a total organic carbon analyzer. The calibration curve used in the total organic carbon analyzer is prepared using a standard solution.

As a total organic analyzer for measuring the concentration (low concentration) of TOC, a device is considered for preparing a low-concentration test solution inside the total organic carbon analyzer (see, for example, Patent Document 1)

### PRIOR ART DOCUMENTS

### Patent Documents

Patent Document 1: JP 3265830

### SUMMARY OF THE INVENTION

### Problem to be Solved by the Invention

Commonly used calibration curves are prepared using standard solutions in the high TOC concentration range. If a calibration curve created using a standard solution in the high TOC concentration range is used to measure a TOC concentration having a low concentration, the percentage of error in the high concentration range will be greater than in the low concentration range. This increased percentage of error prevents the acquisition of an accurate TOC concentration. Therefore, in a total organic carbon analyzer that measures the concentration of TOC in ultrapure water, it is necessary to create a calibration curve using a standard solution with a concentration equivalent to the concentration of TOC in ultrapure water (low concentration). On the other hand, the lower the concentration of the standard solution, the greater the influence of contamination from the environment and human error in the preparation of the standard solution. The device described in Patent Document 1 is a device that obtains low organic concentrations by distilling the sample. This process is time-consuming and expensive because it requires equipment such as combustion tubes and furnaces and further requires the preparation of specialized equipment.

The purpose of the present invention is to provide a calibration curve solution production system, a measurement system, and a calibration curve solution production method that can easily create a calibration curve capable of determining more accurate TOC concentrations.

### Means for Solving the Problem

A calibration curve solution production system of the invention is a calibration curve solution production system that produces and supplies a calibration curve solution for creating a calibration curve to a measurement device that uses the calibration curve to measure the total organic carbon concentration of a liquid to be analyzed, comprising:

a first feed line that conducts diluent to the measurement device;

a second feed line that conducts a standard solution to the first feed line; and

a mixing unit that is provided in the first feed line to dilute the standard solution by mixing the diluent with the standard solution, wherein:

the calibration curve solution production system supplies to the measurement device a mixture of the diluent and the standard solution mixed in the mixing unit as the calibration curve solution.

A measurement system of the invention is a measurement system, comprising:

a calibration curve solution production system of the invention is a calibration curve solution production system that produces and supplies a calibration curve solution for creating a calibration curve to a measurement device that uses the calibration curve to measure the total organic carbon concentration of a liquid to be analyzed, comprising:

a first feed line that conducts diluent to the measurement device;

a second feed line that conducts a standard solution to the first feed line; and

a mixing unit that is provided in the first feed line to dilute the standard solution by mixing the diluent with the standard solution, wherein: the calibration curve solution production system supplies to the measurement device a mixture of the diluent and the standard solution mixed in the mixing unit as the calibration curve solution; and

a measurement device.

A calibration curve solution production method of the invention is a calibration curve solution production method for producing and supplying a calibration curve solution for creating a calibration curve to a measurement device that uses the calibration curve to measure the total organic carbon concentration of a liquid to be analyzed, the method comprising:

a process for diluting a standard solution by mixing a diluent conducted by a first feed line to the measurement device and a standard solution conducted to the first feed line from a second feed line that supplies the standard solution; and

a process for supplying to the measurement device a mixed solution of the diluent and the standard solution as the calibration curve solution.

### Advantageous Effects of the Invention

In this invention, a calibration curve can be easily created that enables more accurate determination of TOC concentrations.

### BRIEF DESCRIPTION OF THE DRAWINGS

FIG. 1 is a diagram showing an embodiment of the calibration curve solution production system of the present invention.

FIG. 2 is a diagram showing an example of the method by which the correction unit shown in FIG. 1 corrects a calibration curve.

FIG. 3 is a diagram showing an example of output values when a calibration curve is created in a high TOC concentration range.

FIG. 4 is a diagram showing an example of output values when a calibration curve is created in a low TOC concentration range. FIG. 5 is a diagram showing an example of the application of the calibration curve solution production system of the present invention.

### DESCRIPTION OF THE EMBODIMENTS

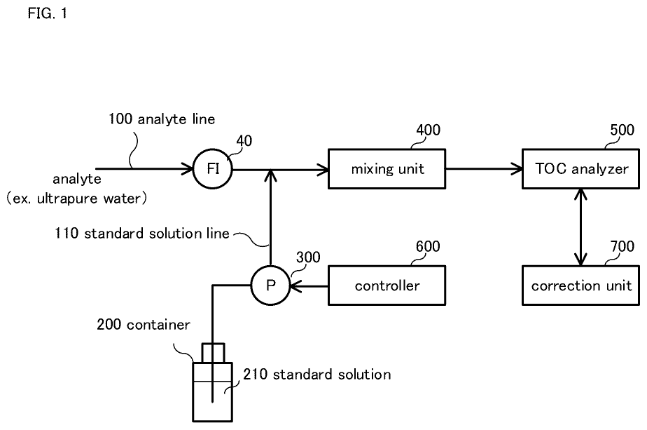

An embodiment of the invention is next described with reference to the drawings. FIG. 1 is a diagram showing an embodiment of the calibration curve solution production system of the present invention. As shown in FIG. 1, the calibration curve solution production system in this embodiment includes analyte line 100, standard solution line 110, pump 300, mixing unit 400, and controller 600. The calibration solution mixed in mixing unit 400 of this calibration curve solution production system is supplied to TOC analyzer 500. Correction unit 700 may be connected to TOC analyzer 500, or alternatively, correction unit 700 may be provided inside TOC analyzer 500. Analyte line 100 is also equipped with flow meter 40 that measures the flow rate of the analyte being conducted to analyte line 100. TOC analyzer 500 may be included as one component of the calibration curve solution production system.

Analyte line 100 is the first feed line that conducts the analyte (e.g., ultrapure water in this case) to TOC analyzer 500. The ultrapure water passed to TOC analyzer 500 can be, for example, at least a portion of the ultrapure water supplied to the point of use from an ultrapure water production system equipped with a pre-treatment apparatus, a primary pure water production apparatus, and a secondary pure water production apparatus (subsystem), or from ultrapure water stored in a specified storage tank. The analyte solution conducted through analyte line 100 is used as a diluent to dilute standard solution 210, which is described below. The diluent that is conducted to analyte line 100 is not limited to the analyte, but can also be ultrapure water or other liquids that are not subject to analysis. In this case, the liquid to be analyzed and the diluent, which is different from the liquid to be analyzed, may be supplied from different pipes.

Pump 300 pumps standard solution 210 stored in container 200. In this embodiment, the performance of pump 300 must have a stable and accurate pumping rate. Pump 300 should have low TOC elution from parts that contact liquid. Pump 300 is preferably but not limited to, for example, a double-plunger type. Pump 300 can be any device as long as it is equipped with the same function as a pump. The pumping rate at which pump 300 pumps standard solution 210 from container 200 (in other words, the pumping rate at which pump 300 pumps standard solution 210 from container 200 to analyte line 100 via the second feed line, i.e., standard solution line 110) is controlled according to the feed rate at which analyte line 100 conducts ultrapure water and the TOC concentration of standard solution 210. This control is next described. Standard solution 210 may be prepared by diluting a standard solution (potassium hydrogen phthalate) linked with an international standard. Standard solution 210 may be prepared by diluting an organic matter such as urea solution actually contained in ultrapure water. Standard solution line 110 conducts standard solution 210 that is pumped using pump 300 to analyte line 100.

Mixing unit 400 mixes the ultrapure water passed through analyte line 100 with standard solution 210 pumped by pump 300. Thus, in mixing unit 400, standard solution 210 pumped by pump 300 is diluted with ultrapure water passed from analyte line 100. A mixing coil may be used as mixing unit 400. The liquid mixed produced by mixing unit 400 is supplied to TOC analyzer 500 as a calibration curve solution.

Controller 600 controls the pumping rate of standard solution 210 (the amount of standard solution 210 supplied from pump 300) by means of pump 300. Based on the feed rate of ultrapure water and the TOC concentration of standard solution 210, controller 600 controls the feed rate of standard solution 210 by pump 300 such that the TOC concentration of the liquid after mixing by mixing unit 400 is below a predetermined value. For example, if the feed rate (flow rate) of ultrapure water through analyte liquid line 100 is 1000 mL/min, the TOC concentration of standard solution 210 is 100 ppb-C, and the TOC concentration of the liquid after mixing by mixing unit 400 is in the low concentration range (0.1 ppb-C+ultrapure TOC concentration of pure water), controller 600 controls pump 300 such that pump 300 pumps standard solution 210 at a pumping rate of 1 mL/min. Thus, controller 600 controls the pumping rate at which pump 300 pumps standard solution 210 from container 200 to less than a predetermined percentage (e.g., 1/100th) of the feed rate at which analyte line 100 conducts ultrapure water. This makes the dilution factor of standard solution 210 using ultrapure water more than a predetermined factor (e.g., 100 times). As standard solution 210 is diluted, a higher dilution factor will result in a smaller effect of any contamination or concentration error in standard solution 210. This approach allows the preparation of standard solutions with more accurate concentrations. Standard solution 210 may be used with a TOC concentration higher than 100 ppb-C. In that case, controller 600 controls the rate at which pump 300 pumps standard solution 210 at a rate lower than 1 mL/min. If the rate (performance) at which pump 300 pumps standard solution 210 is a fixed value, the TOC concentration of standard solution 210 is adjusted so that the TOC concentration of the solution after mixing by mixing unit 400 will be below a predetermined value. Controller 600 is provided with at least the function of stopping or activating the pumping operation of pump 300. Controller 600 may input information based on operations received from the outside and then perform control based on the input information. Controller 600 may also receive signals transmitted from other devices and perform control based on the information indicated by the received signals. Controller 600 may also perform predetermined processing on input information or information indicated by received signals and then effect control according to the results of that processing.

TOC analyzer 500 is a measurement device that measures the total organic carbon concentration of a liquid to be analyzed. TOC analyzer 500 has the function of performing organic decomposition of the supplied liquid and outputting the electrical conductivity and specific resistance values before and after the decomposition as response values. TOC analyzer 500 also has the function of creating a calibration curve to calculate the total organic carbon concentration based on the difference in the response values (output values). TOC analyzer 500 decomposes the organic matter in the calibration curve solution mixed in mixing unit 400 and measures the electrical conductivity or specific resistance of the liquid in which the organic matter has been decomposed. TOC analyzer 500 uses the calibration curve described above to calculate the total organic carbon concentration of the liquid based on the measured electrical conductivity or specific resistance. TOC analyzer 500 is a continuous total organic carbon analyzer that performs periodic measurements. The period may be a predetermined period or may also be an externally configurable period. A continuous type is one in which TOC analyzer 500, once allowed to start operating, continues to measure at each predetermined timing unless an instruction is input to end the operation. TOC analyzer 500 is a measurement device with a measurement range of 0.1 to 500 ppb-C for the total organic carbon concentration. The measurement system consists of the TOC analyzer 500 and the calibration curve solution production system shown in FIG. 1.

Correction unit 700 corrects the calibration curve created by TOC analyzer 500 using a standard addition method based on first and second output values. The first output value is the value measured by TOC analyzer 500 in the absence of pump 300 (or when controller 600 has stopped pump 300 from pumping standard solution 210). The second output value is a value measured by TOC analyzer 500 in a state in which pump 300 has been activated by controller 600 and is pumping. Specifically, correction unit 700 corrects the calibration curve created by TOC analyzer 500 by adding the total organic carbon concentration of ultrapure water obtained using the standard addition method to the total organic carbon concentration of standard solution 210 pumped by pump 300 and diluted with ultrapure water. Correction unit 700 may be provided in TOC analyzer 500.

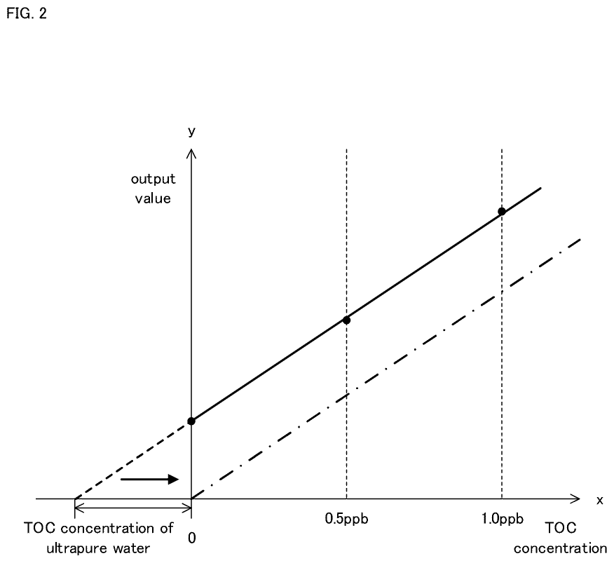

FIG. 2 is a diagram showing an example of the method by which the correction unit shown in FIG. 1 corrects a calibration curve. In FIG. 2, the x-axis indicates the TOC concentration of standard solution 210 pumped by pump 300 (“0” indicates no pumping of standard solution 210 by pump 300). The y-axis indicates the output value of TOC analyzer 500. Correction unit 700 uses the values output by TOC analyzer 500 for a total of at least two TOC concentrations, one point being when the concentration added by pump 300 is “0” (first concentration) and one point being when the concentration added by pump 300 is not “0” (second concentration). Correction unit 700 preferably uses values output by TOC analyzer 500 for a total of three or more TOC concentrations: one point being when the concentration added by pump 300 is “0” and two or more points being when the concentration added by pump 300 is not “0.” On the graph shown in FIG. 2, the three output values are plotted: when the concentration added by pump 300 is “0” (operation of pump 300 is stopped), when the TOC concentration is 0.5 ppb-C after dilution of standard solution 210 pumped by pump 300, and when the TOC concentration is 1.0 ppb-C. The graph in FIG. 2 shows a solid line connecting the three plotted points. If the concentration added by pump 300 is “0” (x=0), the value measured by TOC analyzer 500 will not be “0” (y=0) because the ultrapure water from analyte line 100 also contains TOC. Therefore, correction unit 700 uses the standard addition method to extend the solid line (represented by a dashed line) and thus find the intersection of the dashed line and the x-axis. The concentration corresponding to the distance between the coordinates of “0” and the intersection of the dashed line and the x-axis is the TOC concentration of ultrapure water. Correction unit 700 shifts the calibration curve between the solid and dashed portions in the positive direction of the x-axis by the TOC concentration of this ultrapure water. The shifted straight line is represented by a dash-dotted line. This dash-dotted line is the corrected calibration curve.

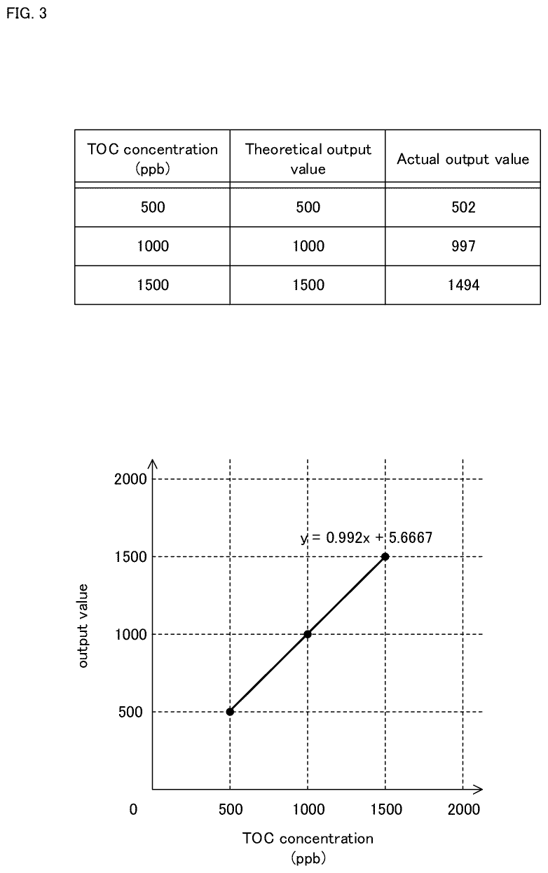

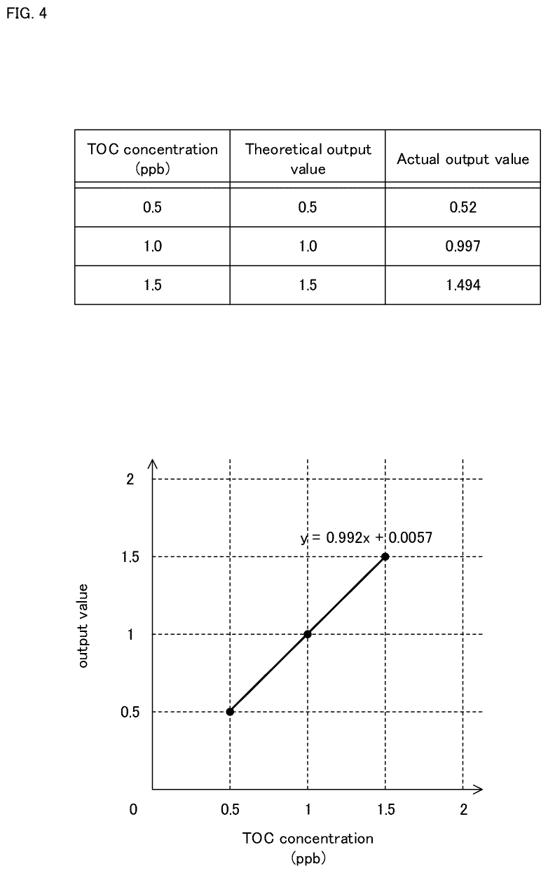

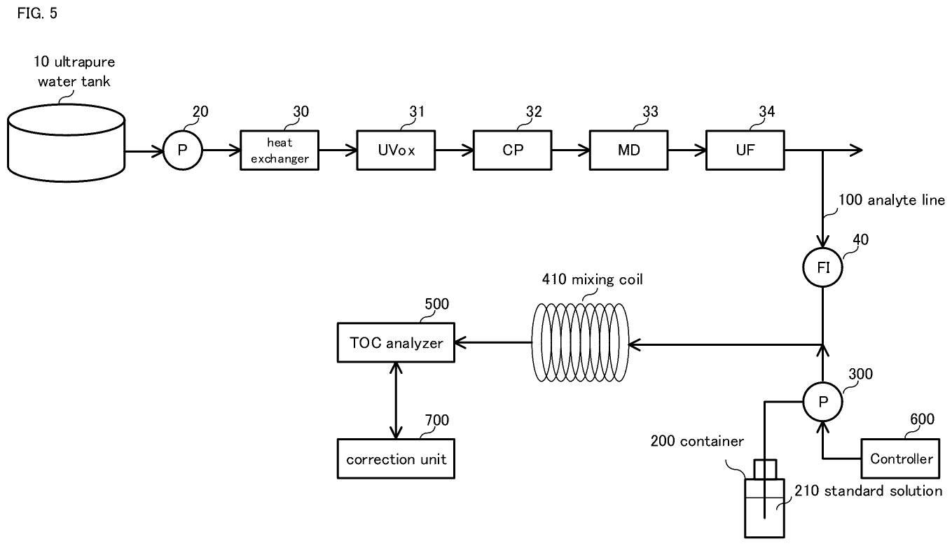

FIG. 3 is a diagram showing an example of output values when a calibration curve is created in the high TOC concentration range. The output values shown in FIG. 3 are the electrical conductivity, specific resistance, or other values, including but not limited to values measured by TOC analyzer 500 to calculate the TOC concentration. These output values are the same as in FIG. 4 described below. FIG. 3 shows a case in which the measurement error of the device is within 1%. As shown in FIG. 3, the theoretical output value is “500” when the TOC concentration of the standard solution is 500 ppb-C, but the actual output value is “502.” The theoretical output value is “1000” when the TOC concentration of the standard solution is 1000 ppb-C, but the actual output value is “997.” The theoretical output value is “1500” when the TOC concentration of the standard solution is 1500 ppb-C, but the actual output value is “1494.” These results are shown graphically in the lower portion of FIG. 3. The equation (calibration curve) relating the TOC concentration (x) to the output value (y) is y=0.992x +5.6667. From this calibration curve, the output value when the TOC concentration is 1 ppb-C, which is a low concentration, is calculated to be “6.6587,” which indicates a large error. Thus, if output values obtained from low-concentration measurements are converted using a calibration curve prepared using a high-concentration standard solution, the measurement value obtained by the conversion will be an unreliable value.

FIG. 4 is a diagram showing an example of output values when a calibration curve is created in the low TOC concentration range. FIG. 4 shows a case in which the measurement error of the device is within 1%. As shown in FIG. 4, the theoretical output value is “0.5” when the TOC concentration of the standard solution is 0.5 ppb-C, but the actual output value is “0.52.” The theoretical output value is “1.0” when the TOC concentration of the standard solution is 1.0 ppb-C, but the actual output value is “0.997.” The theoretical output value is “1.5” when the TOC concentration of the standard solution is 1.5 ppb-C, but the actual output value is “1.494.” These results are shown graphically in the lower portion of FIG. 4. The equation (calibration curve) relating the TOC concentration (x) to the output value (y) is y=0.992x+0.0057. From this calibration curve, the output value when the TOC concentration is 1 ppb-C, which is a low concentration, is calculated to be “0.9977,” which is a small error. Thus, if output values obtained by measurement are converted using a calibration curve prepared using a standard solution near the concentration to be measured, the measurement value obtained by the conversion will be a highly reliable value.

Thus, calibration curves for a measurement device used to measure low TOC concentrations must be prepared using low-concentration standard solutions in order to ensure that the measurements obtained using the calibration curves will be reliable. In general, however, standard solutions may be subject to contamination by the environment from the time they are prepared until they are used. For example, assuming an increase of 5 ppb-C in a TOC concentration due to contamination from the environment, even if a standard solution with a low concentration of 5 ppb-C is prepared, the TOC concentration will be 10 ppb-C at the time of use due to the environment. The percentage increase in TOC concentration is 100%. On the other hand, if a standard solution with a commonly used TOC concentration of 250 ppb-C is prepared, the TOC concentration will be 255 ppb-C at the time of use due to the environment. The percentage increase in TOC concentration would be 2%. Thus, it is preferable to prepare and use a low-concentration standard solution. However, preparation of low-concentration standard solutions is difficult due to the large concentration error. Therefore, a standard solution with a commonly used TOC concentration must be used during preparation and this standard solution must then be diluted to a lower concentration when being supplied to the measurement device. Therefore, using this invention, a standard solution with a TOC concentration equivalent to a commonly used TOC concentration is mixed with ultrapure water by adjusting the feed rate at which ultrapure water is conducted through the analyte line and the rate at which the standard solution is pumped. The liquid is then diluted so that the TOC concentration of the liquid after mixing is low and supplied inline to the measurement device. Furthermore, the calibration curve is corrected using the TOC concentration of the analyte solution obtained by the standard addition method to account for the TOC concentration in ultrapure water that is mixed with the standard solution. This allows the creation of calibration curves with low measurement error in measuring TOC concentrations in the low-concentration range (e.g., 1 ppb-C or lower).

FIG. 5 is a diagram showing an example of the application of the calibration curve solution production system of the present invention. The example shown in FIG. 5 includes ultrapure water tank 10, pump 20, heat exchanger 30, UV oxidizer 31, non-regenerative ion exchange device 32, membrane degasser 33, ultrafiltration device 34, flow meter 40, analyte line 100, container 200 in which standard solution 210 is stored, pump 300, controller 600, mixing coil 410, TOC analyzer 500, and correction unit 700.

Ultrapure water is supplied from ultrapure water tank 10 to heat exchanger 30 using pump 20, and from heat exchanger 30, the water is conducted to analyte line 100 via UV oxidizer 31, nonregenerative ion exchange device 32, membrane degasser 33, and ultrafiltration device 34. Flow meter 40 is provided in analyte line 100. The treatment in each of the water treatment apparatuses, i.e., heat exchanger 30, UV oxidizer 31, nonregenerative ion exchange device 32, membrane degasser 33, and ultrafiltration device 34, is the same as in a typical water treatment system.

Flowmeter 40, container 200, standard solution 210, pump 300, controller 600, TOC analyzer 500, and correction unit 700 are each the same as the components shown in FIG. 1. Mixing coil 410 corresponds to mixing unit 400 shown in FIG. 1. Mixing coil 410 mixes the ultrapure water passed through analyte line 100 with standard solution 210 pumped by pump 300 to dilute standard solution 210.

Although each component has been described individually above, the configuration may be freely combined, or these components may be shared by one or more devices. For example, correction unit 700 may be provided in TOC analyzer 500 as described above. Controller 600 and correction unit 700 may be provided in TOC analyzer 500. Controller 600 and correction unit 700 may be provided in a device separate from TOC analyzer 500, and the device and TOC analyzer 500 may communicate with each other to exchange signals. In addition, the system may be equipped with ordinary ultrapure water production devices or water treatment devices (e.g., ultrafiltration devices).

While the present invention has been described above with reference to embodiments, the present invention is not limited to the above embodiments. Various changes within the scope of the present invention that will be understood by those skilled in the art can be made in the configuration and details of the present invention.

This application claims priority based on JP 2022-083008 filed on May 20, 2022, all disclosures of which are incorporated herein.

1. A calibration curve solution production system that produces and supplies a calibration curve solution for creating a calibration curve to a measurement device that uses the calibration curve to measure the total organic carbon concentration of a liquid to be analyzed, comprising:
a first feed line that conducts diluent to the measurement device;
a second feed line that conducts a standard solution to the first feed line; and
a mixing unit that is provided in the first feed line to dilute the standard solution by mixing the diluent with the standard solution, wherein:
the calibration curve solution production system supplies to the measurement device a mixture of the diluent and the standard solution mixed in the mixing unit as the calibration curve solution.

2. The calibration curve solution production system according to claim 1, further comprising:
a pump that is provided in the second feed line to supply the standard solution to the first feed line; and
a controller that controls the supply rate of the pump, wherein:
the controller controls the pump to keep the total organic carbon concentration of the calibration curve solution below a predetermined value based on a feed rate of the diluent in the first feed line and the total organic carbon concentration of the standard solution.

3. The calibration curve solution production system according to claim 2, wherein:
the predetermined value is 100 ppb-C.

4. The calibration curve solution production system according to claim 2, wherein:
the controller controls the pump so that the standard solution is supplied to the first feed line at a feed rate less than 1/100th of the feed rate of the diluent in the first feed line.

5. The calibration curve solution production system according to claim 2, further comprising:
a correction unit that corrects the calibration curve created by the measurement device based on the total organic carbon concentration of the diluent obtained using a standard addition method based on a first concentration measured by the measuring device when the diluent is being conducted to the measuring device while the pump is stopped by the controller, and a second concentration measured by the measuring device when the calibration curve solution is being conducted to the measuring device while with the pump is activated by the controller.

6. The calibration curve solution production system according to claim 5, wherein:
the correction unit corrects the calibration curve created by the measuring device by adding the total organic carbon concentration of the diluent obtained using the standard addition method to the total organic carbon concentration of the standard solution diluted using the diluent when the pump is activated.

7. The calibration curve solution production system according to claim 1, wherein:
the calibration curve solution production system produces a calibration curve solution for creating a calibration curve for a measurement device that decomposes organic matter in a liquid mixed by the mixing unit, measures the electrical conductivity or specific resistance of the liquid in which the organic matter was decomposed, and calculates the total organic carbon concentration of the liquid based on the measured electrical conductivity or specific resistance.

8. The calibration curve solution production system according to claim 1, wherein:
the measurement range of total organic carbon concentration in the measurement device is from 0.1 to 500 ppb-C.

9. A measurement system, comprising:
the calibration solution production system according to claim 1; and
a measurement device.

10. A calibration curve solution production method for producing and supplying a calibration curve solution for creating a calibration curve to a measurement device that uses the calibration curve to measure the total organic carbon concentration of a liquid to be analyzed, the method comprising:
diluting a standard solution by mixing a diluent conducted by a first feed line to the measurement device and a standard solution conducted to the first feed line from a second feed line that supplies the standard solution; and
supplying to the measurement device a mixed solution of the diluent and the standard solution as the calibration curve solution.
